(19) United States
(12) Patent Application Publication
(10) Pub. No.: US 2024/0013884 A1
(43) Pub. Date: Jan. 11, 2024

(54) REMOVABLE TREATMENT MANAGEMENT APPARATUS AND TREATMENT MANAGEMENT SYSTEM

(71) Applicant: Parnell Technologies Pty. Ltd., Alexandria (AU)

(72) Inventors: David Snapp, Kansas City, MO (US); Kelly Lynch, Kansas City, MO (US); Nora Schrag, Manhattan, KS (US); Alan Bell, Sydney (AU); Brad McCarthy, Sydney (AU); Nicholas Iles, Brisbane (AU); Thomas Blandino, Cottage Grove, WI (US); Ian Janik, Oregon, WI (US); Michael Naughton, Oconomowoc, WI (US); Emily Warner, Stoughton, WI (US)

(21) Appl. No.: 18/349,760

(22) Filed: Jul. 10, 2023

Related U.S. Application Data
(60) Provisional application No. 63/368,136, filed on Jul. 11, 2022.

(51) Int. Cl. G16H 20/17; G16H 40/67; A61D 7/00; A61D 1/02; A01K 11/00

(52) U.S. Cl. CPC G16H 20/17; G16H 40/67; A61D 7/00; A61D 1/025; A01K 11/006; A01K 11/004

(57) ABSTRACT

A treatment management apparatus for managing treatment administered to livestock is provided. The treatment management apparatus may comprise a wireless communication interface configured to receive, from a user system, treatment data representing an animal to be treated and a treatment to be administered; an animal detector configured to detect placement of the treatment management apparatus against a surface of the animal; and an actuation monitor configured to detect actuation of a treatment initiation component to initiate administration of the treatment of the animal; wherein the wireless communication interface is further configured to send, to the user system, administration data regarding the administration of the treatment, wherein the administration data represents one or more events associated with the animal detector and the actuation monitor.

### RELATED APPLICATIONS

The present application claims priority to U.S. Provisional Patent Application No. 63/368,136, filed Jul. 11, 2022, the contents of which are incorporated by reference herein and made part of this specification.

### BACKGROUND

### Field

This disclosure relates generally to treatment of livestock, and more particularly to a removable treatment management apparatus and computer-based treatment management system.

### Description of the Related Art

Treatments, such as hormones, antibiotics, vaccines, and the like, can be administrated to livestock in a variety of ways. In a common scenario, animals are gathered in a facility where they are visually identified by ID number or automatically identified by electronic or wireless means (e.g., using a wand to scan a tag). Once identified, a corresponding treatment is determined and administered (e.g., by injection). A record of the treatment may be kept, such as a written entry on a form or an electronic record entered into a computing system.

### SUMMARY

In some aspects, the techniques described herein relate to a treatment management apparatus for managing treatment administered to livestock, the treatment management apparatus including: a wireless communication interface configured to receive, from a user system, treatment data representing an animal to be treated and a treatment to be administered; an animal detector configured to detect placement of the treatment management apparatus against a surface of the animal; and an actuation monitor configured to detect actuation of a treatment initiation component to initiate administration of the treatment of the animal; wherein the wireless communication interface is further configured to send, to the user system, administration data regarding the administration of the treatment, wherein the administration data represents one or more events associated with the animal detector and the actuation monitor.

In some aspects, the techniques described herein relate to a treatment management apparatus, further including a treatment tag detector configured to read a treatment tag affixed to a vial.

In some aspects, the techniques described herein relate to a treatment management apparatus, further including a vial detector configured to detect installation of the vial, wherein the treatment tag detector is configured to activate the treatment tag detector in response to detecting installation of the vial.

In some aspects, the techniques described herein relate to a treatment management apparatus, further including an indicator component configured to indicate whether the vial includes the treatment represented by the treatment data received from the user system.

In some aspects, the techniques described herein relate to a treatment management apparatus, further including a securement portion configured to removably attach to a treatment injection device, wherein the actuation monitor is configured to detect movement of an actuator of the treatment injection device.

In some aspects, the techniques described herein relate to a treatment management apparatus, wherein the securement portion includes a first sidewall and a second sidewall forming a channel, wherein to removably attach the treatment management apparatus to the treatment injection device, the treatment injection device is inserted into the channel.

In some aspects, the techniques described herein relate to a treatment management apparatus, wherein an interior surface of the first sidewall includes a securement structure configured to interact with a registration structure of the treatment injection device, wherein interaction of the securement structure with the registration structure reduces slippage of the treatment management apparatus.

In some aspects, the techniques described herein relate to a treatment management apparatus, wherein the actuation monitor pivots at a pivot point when the actuator of the treatment injection device is moved.

In some aspects, the techniques described herein relate to a treatment management apparatus, wherein the actuation monitor includes a trigger object, wherein an inner surface of the securement portion includes an actuation sensor configured to detect the trigger object.

In some aspects, the techniques described herein relate to a treatment management apparatus, wherein the actuation sensor includes a Hall effect sensor.

In some aspects, the techniques described herein relate to a treatment management apparatus, wherein the inner surface of the securement portion includes a second actuation sensor configured to detect the trigger object, wherein detection of the trigger object by the actuation sensor corresponds to actuation of the actuator of the treatment injection device, and wherein detection of the trigger object by the second actuation sensor corresponds to positioning of the actuator of the treatment injection device in an unactuated position.

In some aspects, the techniques described herein relate to a treatment management apparatus, wherein the wireless communication interface is configured to generate the treatment data in response to detecting, using the actuation monitor, movement of the actuator of the treatment injection device.

In some aspects, the techniques described herein relate to a treatment management apparatus, wherein the animal detector includes a trigger object, wherein an inner surface of the securement portion includes an animal contact sensor configured to detect the trigger object.

In some aspects, the techniques described herein relate to a treatment management apparatus, wherein the wireless communication interface is configured to generate the treatment data in response to detecting, using the actuation monitor, the movement of the actuator of the treatment injection device and detecting, using the animal detector, the treatment management apparatus against a surface of the animal.

In some aspects, the techniques described herein relate to a treatment management apparatus, wherein the wireless communication interface is configured to generate second treatment data in response to detecting, using the actuation monitor, the movement of the actuator of the treatment injection device and failing to detect, using the animal detector, the treatment management apparatus against a surface of the animal, wherein the second treatment data represents one of a priming operation or a wasted treatment administration operation.

In some aspects, the techniques described herein relate to a treatment management apparatus, wherein the treatment management apparatus is fixable, removeable, and reusable.

In some aspects, the techniques described herein relate to a system including: a treatment management apparatus for use with a treatment injection device for managing treatment administered to livestock using the treatment injection device, the treatment management apparatus including: an actuation monitor configured to detect movement of an actuator of a treatment injection device; a treatment tag detector configured to read a treatment tag affixed to a vial installed in the treatment injection device; and a vial detector configured to detect installation of the vial into the treatment injection device; and an executable treatment administration manager configured to cause a user system to communicate with the treatment management apparatus.

In some aspects, the techniques described herein relate to a system, wherein the executable treatment administration manager is further configured to cause the user system to communicate with an application server regarding treatment regimens to be implemented using the treatment management apparatus.

In some aspects, the techniques described herein relate to a system, further including an animal identification device configured to read a tag affixed to an animal and transmit, to the user system, animal identification data read from the tag.

In some aspects, the techniques described herein relate to a system, wherein the treatment management apparatus further includes a securement portion configured to removably attach to the treatment injection device.

In some aspects, the techniques described herein relate to a system, wherein the treatment management apparatus is fixable, removeable, and reusable.

In some aspects, the techniques described herein relate to a treatment management apparatus for use with a treatment injection device for managing treatment administered to livestock using the treatment injection device, the treatment management apparatus including: a securement portion configured to be fixed to a treatment injection device; an actuation monitor configured to detect movement of an actuator of the treatment injection device; and an electronic portion including: a treatment tag detector configured to read a treatment tag affixed to a vial installed in the treatment injection device; a vial detector configured to detect installation of the vial into the treatment injection device, wherein the treatment tag detector is configured to activate the treatment tag detector in response to detecting installation of the vial; and a wireless communication interface configured to: receive, from a user system, treatment data representing an animal to be treated and a treatment to be administered; and send, to the user system, administration event data representing one or more events associated with the animal and the treatment.

In some aspects, the techniques described herein relate to a removable treatment management apparatus for use with a treatment injection device for managing treatment administered to livestock using the treatment injection device, the removable treatment management apparatus including: a securement portion configured to removably attach to a treatment injection device; an actuation monitor configured to detect movement of an actuator of the treatment injection device; and an electronic portion including: a treatment tag detector configured to read a treatment tag affixed to a vial installed in the treatment injection device; a vial detector configured to detect installation of the vial into the treatment injection device, wherein the treatment tag detector is configured to activate the treatment tag detector in response to detecting installation of the vial; and a wireless communication interface configured to: receive, from a user system, treatment data representing an animal to be treated and a treatment to be administered; and send, to the user system, administration event data representing one or more events associated with the animal and the treatment.

In some aspects, the techniques described herein relate to a system including: a removable treatment management apparatus for use with a treatment injection device for managing treatment administered to livestock using the treatment injection device, the removable treatment management apparatus including: a securement portion configured to removably attach to a treatment injection device; an actuation monitor configured to detect movement of an actuator of the treatment injection device; and a treatment tag detector configured to read a treatment tag affixed to a vial installed in the treatment injection device; and a vial detector configured to detect installation of the vial into the treatment injection device; and an executable treatment administration manager configured to cause a user system to communicate with the removable treatment management apparatus.

### BRIEF DESCRIPTION OF THE DRAWINGS

Embodiments of various inventive features will now be described with reference to the following drawings. Throughout the drawings, reference numbers may be re-used to indicate correspondence between referenced elements. The drawings are provided to illustrate example embodiments described herein and are not intended to limit the scope of the disclosure.

FIG. 1 is a diagram of a user system in communication removable treatment management apparatus, an application server, and an animal identification device, according to some embodiments.

FIGS. 2 and 3 are diagrams of a removable treatment management apparatus removably attached to a treatment injection device according to some embodiments.

FIGS. 4, 5A, and 5B are diagrams of a removable treatment management apparatus according to some embodiments.

FIG. 6 is a diagram of an inner surface of a removable treatment management apparatus with an actuation monitor according to some embodiments.

FIGS. 7, 8, and 9 are diagrams of a removable treatment management apparatus according to some embodiments.

FIGS. 10A and 10B are diagrams of an animal detector of a removable treatment management apparatus according to some embodiments.

FIG. 11 is a flow diagram of an illustrative routine for managing administration of treatment to an animal using a removable treatment management apparatus removably attached to a treatment injection device according to some embodiments

### DETAILED DESCRIPTION

The present disclosure is directed to a treatment management apparatus and computer-based treatment management system for treatment of livestock, such as cattle.

Cattle and other livestock may be treated with a variety of drugs, such as reproductive hormones, vaccines, antibiotics, and the like. Some treatments are to be provided according to a particular regimen. For example, GONAbreed® (gonadorelin acetate) and estroPLAN® (cloprostenol sodium) from Parnell® in Overland Park, KS are used to synchronize estrous cycles in beef and lactating dairy cows. The drugs are typically administered to cows based on a regimen that specifies dosing and timing of the injections.

Some conventional administration devices, such as needle-based injectors and transdermal/needless systems, are configured to receive or be coupled to a vial or other reservoir containing multiple doses of a drug (e.g., 10 2 ml doses or 20 1 ml doses in a 20 ml vial, 50 2 ml or 100 1 ml doses in a 100 ml vial, etc.). An injection device, such as the Simcro VS Injector from Datamars in Temple, TX, may have a handheld “injector gun” form factor with a trigger-based actuator that causes administration of a dose from a vial through a needle to a target animal. Transdermal and other needless administration devices may be similarly configured in a handled form factor with an actuator that causes administration of a dose from a reservoir to a target animal. In order to determine the proper dose and timing for a particular animal, individual animals may be identified visually by a person or automated visual identification system (e.g., by a number printed on a tag attached to an ear or collar of the animal) or electronically (e.g., by a radio frequency identifier or RFID tag attached to an ear or collar of the animal). Some users may use a computing system to plan the appropriate regimen, track administered doses, and determine adherence to the regimen. Although planning and tracking may be done on such a computing system, the administration and tracking of doses is still a largely manual process. For example, the computing system may maintain generate a target list of animals for proposed dosing, indicating the next dose to be administered (e.g., drug, volume, and timing). However, it is up to the users to ensure that the proper drug is loaded into the injector and that the proper dose is administered. This manual process may be difficult for users to follow when in a livestock facility, around live animals, exposed to the elements, under time pressure, and the like. A mistake at any point in the process can lead to a failure to follow the appropriate regimen for a particular animal, which can affect production. For example, there may be a drag on productivity through loss of estrous synchrony (e.g., due to mistakes in administration of hormones), bacterial infection (e.g., due to mistakes in administration of antibiotics) or failure of immunity (e.g., due to mistakes in administration of vaccines). Moreover, in many conventional systems no record is maintained regarding completed administration of a dose. Those that do allow for record keeping rely on manual entry or confirmation from the user, introducing another source of potential errors.

Some aspects of the present disclosure address some or all of the issues noted above, among others, through use of a removable treatment management apparatus that is removably attachable to an injection device. The removable treatment management apparatus may be configured to provide a friction fit to an injection device. In some embodiments, the removable treatment management apparatus may include one or more securement structures that are sized, shaped, and positioned to interact with corresponding registration structures on the injection device to inhibit or prevent slippage or inadvertent removal of the removable treatment management apparatus from the injection device.

As used herein, the term “removable” when applied to the removable treatment management apparatus refers to a property of being non-permanently fixed to an injection device and to being reusable when reaffixed to the same device or affixed to another device. Thus, the removable treatment management apparatus may be removed (e.g., by hand) without damage to the removable treatment management apparatus or injection device, and therefore may be re-used with the same injection device or another injection device.

The removable treatment management apparatus may include one or more sensors to track operation of the injection device or to otherwise track administration of treatment to animals. In some embodiments, the removable treatment management apparatus may include an actuator monitor that interacts with the actuator of the injection device (e.g., the trigger or other treatment initiation component) and detects when the actuator is partially or completely actuated. In this way, the removable treatment management apparatus can automatically and reliably track the administration of treatment. In some embodiments, the removable treatment management apparatus may include a vial sensor that detects insertion of a vial into the injector device. The vial sensor, or another sensor triggered by detection of a vial by the vial sensor, may determine the drug that is contained within the inserted vial. For example, the vial may include an RFID tag that has been programmed to indicate the particular drug that is contained within the vial, the volume or other measurement of doses contained within the vial, a unique identifier of the vial itself, a batch identifier for a batch to which the vial belongs, other information, or some combination thereof. In this way, the removable treatment management apparatus can track the specific treatment that is administered, the remaining doses available, and the like. The information may be provided to another system (e.g., user device or centralized sever) for purposes of tracking adherence to a treatment regimen, to satisfy regulatory requirements, to provide consumers with information about the treatment history of the source animal of the products being consumed, etc. In some embodiments, the removable treatment management apparatus may include an animal sensor that detects interaction of the removable treatment management apparatus with the surface of an animal. For example, a movable structure of the removable treatment management apparatus may, when pressed against an injection site of the animal, trigger the animal sensor. In this way, actuation of the injection device may be tracked in combination with detection of the animal to distinguish between treatment administration operations and priming or other non-treatment-administration operations. In some embodiments, detection of the animal by the animal sensor may further confirm proper route of administration (e.g., subcutaneous vs. intramuscular), which may vary depending upon the treatment being administered. Such confirmation can serve to ensure that treatment is administered according to regulatory requirements and desired efficacy, for example.

Additional aspects of the present disclosure relate to a computer-based treatment management system that communicates with the removable treatment management apparatus to track usage and interact with a user to manage use of the removable treatment management apparatus. The computer-based treatment management system (also referred to as simply a “treatment management system” for brevity) may maintain, or communicate with a separate system that maintains, records of treatment administrations and details regarding treatment regimens for particular animals. In some embodiments, when the treatment management system receives an indication of a particular animal (e.g., via a wireless communication from an electronic wand interacting with an RFID tag attached to the animal), the treatment management system can communicate to one or more removable treatment management apparatuses the treatment to be administered (e.g., drug, dose, etc.) and the identity of the animal to which the treatment is to be administered. Each removable treatment management apparatus may include a processor to determine whether the vial currently inserted includes the identified treatment, whether the removable treatment management apparatus is configured to provide the indicated dosage, and/or other operational criteria. The removable treatment management apparatus may then provide an indication to the user regarding whether the removable treatment management apparatus is currently set to provide the indicated treatment. In some embodiments, the removable treatment management apparatus may include one or more indicator components, such as a light emitting diode (LED), audible indicator component (e.g., speaker) and/or tactile feedback component (e.g., vibration device), that will indicate to a user whether the removable treatment management apparatus is currently configured to provide the proper treatment to the animal. When the removable treatment management apparatus is used to administer treatment (whether proper or not), the removable treatment management apparatus can communicate back to the treatment management system the drug that was administered, the dose that was administered, the animal to which the dose was administered, and a timestamp reflecting the date and time of administration. In this way, the treatment management system can facilitate indication to a user of the proper injection device to administer the appropriate treatment, and track the treatment that was actually administered, whether planned or not.

Various aspects of the disclosure will now be described with regard to certain examples and embodiments, which are intended to illustrate but not limit the disclosure. Although the examples and embodiments described herein will focus, for the purpose of illustration, on specific devices and operations, one of skill in the art will appreciate the examples are illustrative only, and are not intended to be limiting. In addition, any feature, process, device, or component of any embodiment described and/or illustrated in this specification can be used by itself, or with or instead of any other feature, process, device, or component of any other embodiment described and/or illustrated in this specification.

### Example Treatment Environment

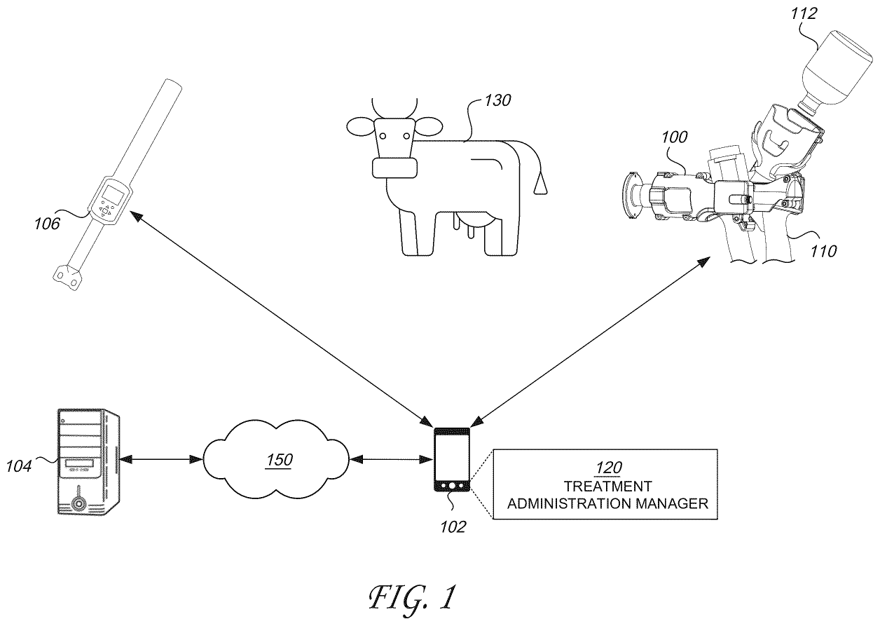

FIG. 1 shows an example environment in which aspects of the present disclosure may be implemented according to some embodiments. As shown, a user system 102 may be in communication with an application server 104, an animal identification device 106, and a removable treatment management apparatus 100. The removable treatment management apparatus 100 may be removably attached to a treatment injection device 110, and may be used to manage administration of treatment from a vial 112 to an animal 130.

In some embodiments, the user system 102 may communicate with the application server 104 via a communication network 150. The communication network 150 (also referred to simply as a “network”) may be a publicly-accessible network of linked networks, possibly operated by various distinct parties, such as the Internet. In some cases, the network 150 may be or include a private network, personal area network, local area network, wide area network, global area network, cable network, satellite network, cellular data network, etc., or a combination thereof, some or all of which may or may not have access to and/or from the Internet. In some embodiments, the user system 102 may communicate with the removable treatment management apparatus 100 and/or animal identification device 106 using relatively short-distance wireless communication methods, such as Wi-Fi, Bluetooth®, Bluetooth® Low Energy (BLE), or the like.

The user system 102 may be any of a wide variety of electronic devices with communication and application execution capabilities, including personal computing devices, laptop computing devices, tablet computing devices, electronic reader devices, wearable computing devices, mobile devices (e.g., cellular and other mobile phones, smart phones, media players, handheld gaming devices, etc.), and various other electronic devices and appliances. The user system 102 may include a treatment administration manager 120 to manage and track treatment administration operations, and to manage and track communication with the removable treatment management apparatus 100, animal identification device 106, and/or application server 104. In some embodiments, the treatment administration manager 120 is or includes application software (e.g., a library of object code or otherwise executable code) to provide the various features.

The application server 104 may be any of a wide variety of computing devices with application execution, data storage, and network communication capabilities for providing a network-based application, such as a web site or server-side of an application with a client-side component executing on user systems 102. In some embodiments, the application server 104 may be may be implemented on one or more host devices, such as blade servers, midrange computing devices, mainframe computers, desktop computers, or any other computing device configured to provide computing services and resources.

The animal identification device 106 may be any of a wide variety of devices with detection and communication capabilities for providing identifying information (also referred to animal identification data) detected from an identification source, such as a barcode, QR code, tag, RFID chip, or the like. In some embodiments, the animal identification device may be a handheld device, such as the AWR-300 Wand from Agrident GmbH in Barsinghausen, Germany.

The removable treatment management apparatus 100 may be a device that is removably attachable to a treatment injection device 110. Generally described, the treatment injection device 110 may be implemented using a handheld injector gun form factor, such as the Datamars Simcro VS Injector. In some embodiments, the removable treatment management apparatus 100 may be removably attached to the treatment injection device 110 by inserting the treatment injection device 110 into a channel formed by a plurality of sidewalls of the removable treatment management apparatus 100. In some embodiments, the sidewalls may be designed to exert a gripping force on the treatment injection device 110. In some embodiments, within the channel (e.g., on the interior surface of one or more sidewalls) may be one or more securement structures that are sized, shaped, and positioned to interact with corresponding registration structures on the treatment injection device 110 to inhibit or prevent slippage or inadvertent removal of the removable treatment management apparatus 100 from the treatment injection device 110. Examples of removable treatment management apparatuses 100, methods of removably attaching to treatment injection devices 110, and features provided by removable treatment management apparatuses 100 are described in greater detail throughout the present disclosure.

With reference to an illustrative embodiment, application server 104 may store treatment regimen data regarding a treatment regimen to which a particular animal 130 is assigned. For example, the regimen may call for a specific dose of medication (e.g., reproductive hormone, vaccine, antibiotic, etc.) at a particular time and/or according to a particular schedule. When a user is preparing to administer medication to one or more animals, the regimen data may be provided by the application server 104 to the treatment administration manager 120 or otherwise to the user system 102. For example, the user system 102 may request current regimen data from the application server 104, and store the regimen data on the user system 102 for use in managing administration of medication.

The treatment administration manager 120 (which may also be referred to simply as an “administration manager” for brevity) may connect to one or more external devices to manage administration of medication. For example, the treatment administration manager 120 may establish a wireless connection to an animal identification device 106 and a removable treatment management apparatus 100. In some embodiments, the treatment administration manager 120 may connect to a plurality of removable treatment management apparatuses 100, and each removable treatment management apparatus 100 may be removably attached to a different treatment injection device 110. The different treatment injection devices 110 may be configured to administer different drugs, administer the same drugs, administer different doses of the drugs, administer the same doses of the drugs, or any combination thereof.

A user may install a vial 112 into a treatment injection device 110 to which a removable treatment management apparatus 100 has been removably attached. Upon installation of the vial 112, the removable treatment management apparatus 100 may transmit, to the treatment administration manager 120, vial data regarding the vial 112. For example, the removable treatment management apparatus 100 may include a vial detection component, such as an inductive sensor configured to detect a metal band on the rim of the vial 112 being properly seated in the treatment injection device 110. When installation of the vial 112 is detected, a vial identification device of the removable treatment management apparatus 100 may be activated to determine the identity and content of the vial 112. For example, the removable treatment management apparatus 100 may include an RFID reader configured to read an RFID tag attached to the vial 112, such as an RFID tag embedded in a label of the vial 112. The RFID tag may provide information regarding the identity and content of the vial 112, such as a unique vial identifier of the vial 112 itself, a treatment identifier of the treatment contained in the vial 112, other information, or some combination thereof. The removable treatment management apparatus 100 may then transmit, to the user system 102, the information regarding the vial 112. In some embodiments, the removable treatment management apparatus 100 may transmit additional and/or alternative information, such as information about the identity of the removable treatment management apparatus 100 (e.g., a unique removable treatment management apparatus 100 identifier), information about the dose that the removable treatment management apparatus 100 is configured to administer, other information, or some combination thereof. Examples of the data that the removable treatment management apparatus 100 provides to the user system 102 are described in greater detail below.

The user (or another user, such as an assistant or team member) may use the animal identification device 106 to identify a particular animal 130 or set of animals to be treated. In some embodiments, each animal 130 may be tagged and assigned a unique animal identifier. For example, an RFID tag may be attached to the animal 130. The animal identification device 106 may obtain an animal identifier from the RFID tag, and provide the animal identifier to the user system 102. In some embodiments, verification of the animal may be manual, such as by visual confirmation of an identification number on a tag attached to the animal 130.

In some embodiments, an animal identification device 106 or features thereof may be integrated into the removable treatment management apparatus 100. However, as this may present a crush risk to a user who would use the removable treatment management apparatus 100 to both detect and read from the RFID tag attached to the animal 130 (e.g., by reading through or next to a head stock) and then administer treatment (e.g., by contacting an injection site), it may be advantageous in some embodiments to keep the animal identification features on a device that is physically separate from the removable treatment management apparatus 100.

The treatment administration manager 120 may determine the treatment to be administered to the animal 130 based on the animal identifier received from the animal identification device 106. In some embodiments, the treatment may be determined based on regimen data that was previously received from the application server 104, or on regimen data that is retrieved form the application server 104 in response to receipt of the animal identifier. In some embodiments, the treatment may be determined based on data maintained by the treatment administration manager 120 or another subsystem of the user system 102 without communication with an application server 104.

The treatment administration manager 120 may transmit treatment data to the removable treatment management apparatus 100. The treatment data may indicate the particular treatment to be administered to the animal 130 identified by the animal identifier. For example, the treatment data may include the treatment identifier for the treatment to be administered, and the animal identifier for the animal to which the treatment is to be administered. If there are multiple removable treatment management apparatuses 100 in communication with the user system 102 and with vials installed into the respective treatment injection devices 110, the treatment administration manager 120 may transmit the treatment data to each of the removable treatment management apparatuses 100 or a subset thereof.

In some embodiments, treatment data may not indicate or be otherwise associated with a particular animal, and may therefore not include or be associated with an animal identifier. For example, treatment may be administered to each animal in a lot, pen, or other group. In such case, doses administered may still be tracked for the group even if not tied to individual animals within the group.

Upon receipt of treatment data from the treatment administration manager 120, a removable treatment management apparatus 100 may determine whether the treatment in the vial 112 currently installed in the treatment injection device 110 to which the removable treatment management apparatus 100 is attached is the treatment identified in the treatment data. If so, the removable treatment management apparatus 100 may indicate to the user that the removable treatment management apparatus 100 may be used to administer the indicated treatment. For example, the removable treatment management apparatus 100 may include an indicator component, such as a light emitting diode (LED) that will illuminate in a particular color (e.g., green) or pattern (solid) to indicate that the removable treatment management apparatus 100 may be used to administer the indicated treatment. As another example, the indicator component may produce an audible notification of a particular tone or pattern, or the indicator component may produce tactile feedback (e.g., vibration) of a particular strength or pattern, to indicate that the removable treatment management apparatus 100 may be used to administer the indicated treatment. Otherwise, if the treatment in the vial 112 currently installed in the treatment injection device 110 to which the removable treatment management apparatus 100 is attached is not the treatment identified in the treatment data, then the removable treatment management apparatus 100 may indicate to the user that the removable treatment management apparatus 100 is not currently configured to administer the indicated treatment. For example, an LED may illuminate in a particular color (e.g., red) or pattern (blinking)—or not illuminate at all—to indicate that the removable treatment management apparatus 100 is not currently configured to administer the indicated treatment. As another example, the indicator component may produce an audible notification of a particular tone or pattern—or no tone at all—or the indicator component may produce tactile feedback (e.g., vibration) of a particular strength or pattern—or no tactile feedback at all—to indicate that the removable treatment management apparatus 100 is not currently configured to administer the indicated treatment.

The user may administer treatment from the treatment injection device 110 with the removable treatment management apparatus 100 attached. For example, the treatment injection device 110 may have an injection gun form factor. The user may place the treatment injection device 110 with the removable treatment management apparatus 100 attached against the skin of the animal 130, and actuate injection of the treatment by pulling a trigger.

The removable treatment management apparatus 100 may generate administration data regarding administration of the treatment to the animal 130, such as data regarding various events that occur during the administration process. In some embodiments, the removable treatment management apparatus 100 may include an animal sensor that detects interaction of the removable treatment management apparatus 100 with the surface of the animal 130. For example, a movable structure of the removable treatment management apparatus 100 may, when pressed against an injection site of the animal 130, trigger the animal sensor. The removable treatment management apparatus 100 may generate data regarding this event. In some embodiments, the removable treatment management apparatus 100 may include an actuator monitor that interacts with the actuator of the injection device (e.g., the trigger) and detects when the actuator is partially or completed actuated. The removable treatment management apparatus 100 may generate data regarding this event. Administration event data regarding these events may be transmitted to the user system 102.

In some embodiments, the treatment administration manager 120 (and/or the removable treatment management apparatus 100 itself) may use the administration event data to determine whether and how much treatment was administered to the animal 130. For example, if the animal sensor was not triggered while the actuator monitor was, then those events may be considered “priming” operations or wasted treatment administration operations. The treatment administration manager 120 may confirm receipt of the administration event data by resending the administration event data, or data derived therefrom, back to the removable treatment management apparatus 100. The removable treatment management apparatus 100 may compare the confirmation message received from the user system 102 to the administration event data sent to the user system 102 and, if they match, the removable treatment management apparatus 100 is free to delete the administration event data and/or data from which the administration event data is derived from memory of the removable treatment management apparatus 100. Examples of administration event data generation, transmission, and processing are described in greater detail below.

As described above, attachment of the removable treatment management apparatus 100 to the treatment injection device 110, together with use of the treatment administration manager 120, can provide a variety of advantageous features that are not practical or possible by using the treatment injection device 110 alone. For example, conventional treatment injection devices 110 and treatment planning and tracking programs are not capable of dynamically providing feedback to users regarding whether the treatment injection device 110 is the proper one to use for a given treatment administration procedure based on automatic detection of vials 112 and treatments contained therein, and on scheduled treatment regimen data. As another example, conventional treatment injection devices 110 and treatment planning and tracking programs are not capable of automatically and accurately tracking full treatment administrations, partial treatment administrations, and priming or wasted treatments. Tracking both priming doses (or other wasted treatments) and doses actually administered to animals (trigger pulls with animals detected) provides greater inventory control and tracking. Also, having a verified administration record for a specific dose of treatment to a specific animal on a specific date provides transparency and adds value to consumers. As a further example, conventional management and tracking can be particularly difficult when multiple (e.g., dozens or hundreds) of animals are to be treated in a row.

The example features and benefits describe herein are illustrative only and are not intended to be limiting, required, or exhaustive. In some embodiments, additional and/or alternative features and benefits may be provided. For example, in some embodiments, a treatment management apparatus may be fixable to a treatment injection device (e.g., via screws, adhesives, etc.) such that it is not easily removable by hand, or not removable without the use of a tool. In addition, although the figures and the description herein focus on use of the removable treatment management apparatus 100 with a needle-based injection device, the figures and description are illustrative only and are not intended to be limiting, required, or exhaustive of the administration devices with which a removable treatment management apparatus 100 may be used. In some embodiments, any, some, or all of the features described herein may be used with needle-free injection devices, such as transdermal devices.

### Example Removable Treatment Management Apparatus

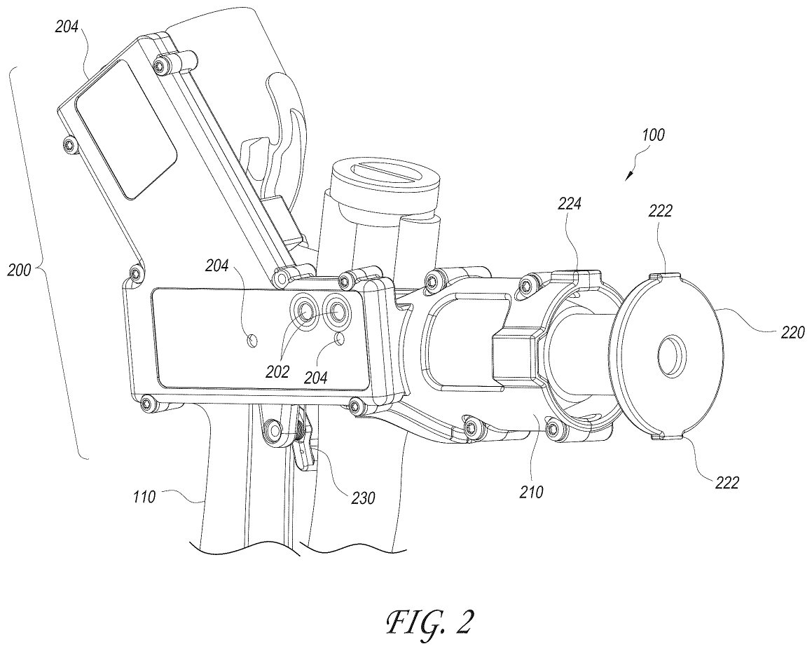

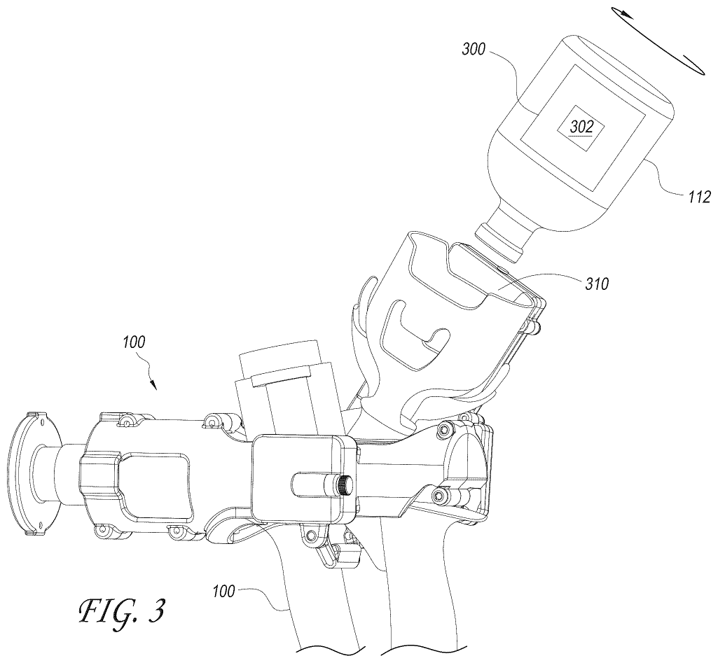

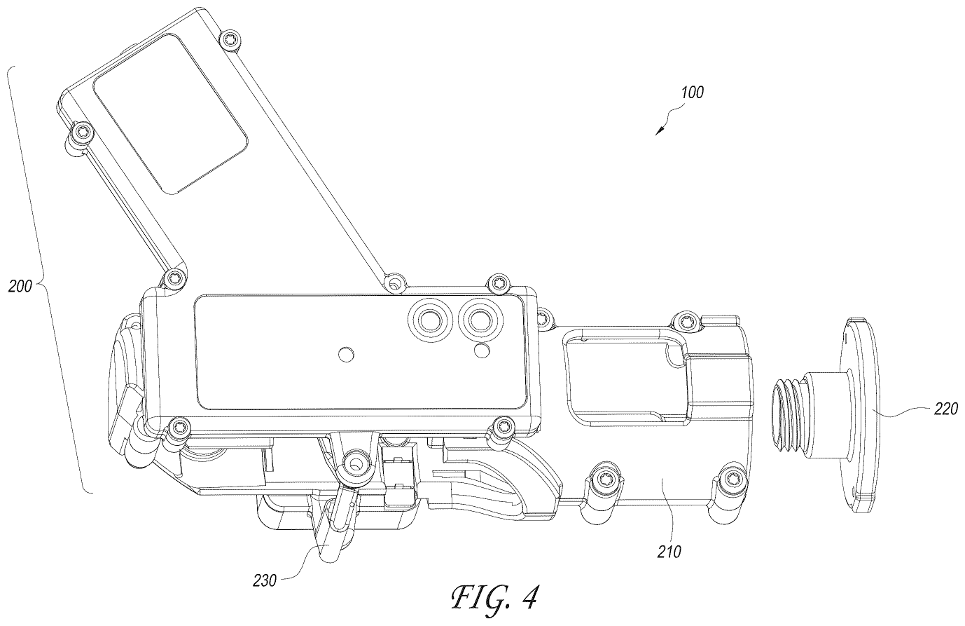

FIGS. 2, 3, 4, 5A, 5B, and 6 are various views of an example of a removable treatment management apparatus 100 according to some embodiments. In FIGS. 2 and 3 the removable treatment management apparatus 100 is attached to a treatment injection device 110. FIGS. 4, 5A, 5B, and 6 illustrate the removable treatment management apparatus 100 without being attached to a treatment injection device 110 in order to provide clear views of various features of the removable treatment management apparatus 100.

As shown, the removable treatment management apparatus 100 includes an electronic portion 200, a securement portion 210, an animal detector 220, and an actuation monitor 230.

The electronic portion 200 may house a printed circuit board (PCB) and/or some other electronic circuitry to facilitate various features described herein. Power for the PCB and/or other components of the removable treatment management apparatus 100 may be provided by a rechargeable and/or replaceable battery that is within the electronic portion 200 or electrically coupled to components of the electronic portion 200. In some embodiments, the electronic portion 200 may include a wireless communication interface, such as a Bluetooth® or Wi-Fi antenna, to communicate with a user system 102. In some embodiments, the electronic portion 200 may include a processor and memory to execute instructions and store data. The processor and/or memory may be integrated with the wireless communication interface, integrated with the PCB external to the wireless communication interface, or included in some other portion of the electronic portion 200.

In some embodiments, the electronic portion 200 may include a treatment tag detector 310, such as an RFID antenna, configured to detect and receive data from RFID tags attached to treatment vials 112. For example, there may be a label 300 affixed to a vial 112. The label 300 may include various information in visible form about the treatment contained in the vial 112, such as the name of the treatment, the recommended dose of the treatment, the size of the vial 112, an expiration date, a lot number, other information, or some combination thereof. In addition, the label 300 may include an embedded treatment tag 302, such as an RFID tag, that is configured to provide various information about the treatment to the treatment tag detector 310, such as a unique identifier of the vial 112, an identifier of the treatment contained in the vial 112, a size of the vial 112, a number of doses of treatment in the vial 112, other information, or some combination thereof. In some embodiments, in order to detect and read the tag 302, the vial 112 is to be rotated such that the label 300, or a portion of the label 300 that includes the treatment tag 302, is adjacent to the treatment tag detector 310. In some embodiments, a vial 112 may include multiple treatment tags 302 to increase the likelihood of a treatment tag 302 being adjacent to the treatment tag detector 310 when the vial 112 is installed into the removable treatment management apparatus 100.

In addition to internal electronic components, the electronic portion 200 may include or be electrically coupled to one or more externally accessible components. In some embodiments, one or more actuator components 202 may be accessible from outside the electronic portion 200 to activate or access various features. For example, the electronic portion 200 may include an actuator component 202 such as a button to power on the removable treatment management apparatus 100, an actuator component 202 such as a button to indicate that the next administration operation is a priming operation rather than administration of treatment to an animal, etc. In some embodiments, one or more indicator components 204 may be viewable from outside the electronic portion 200. For example, an indicator component 204 may be an LED to indicate the power status or wireless communication status of the removable treatment management apparatus 100. As another example, an indicator component 204 may be an LED to indicate the connection status of the removable treatment management apparatus 100 to the user system 102. As a further example, an indicator component 204 may be a visual indicator (e.g., an LED), an audible indicator (e.g., a speaker), or a tactile indicator (e.g., a vibration component) to indicate whether the removable treatment management apparatus 100 is to be used to perform the next administration to the next animal, or whether the removable treatment management apparatus 100 does not have the proper vial 112 installed with the treatment to be administered to the next animal.

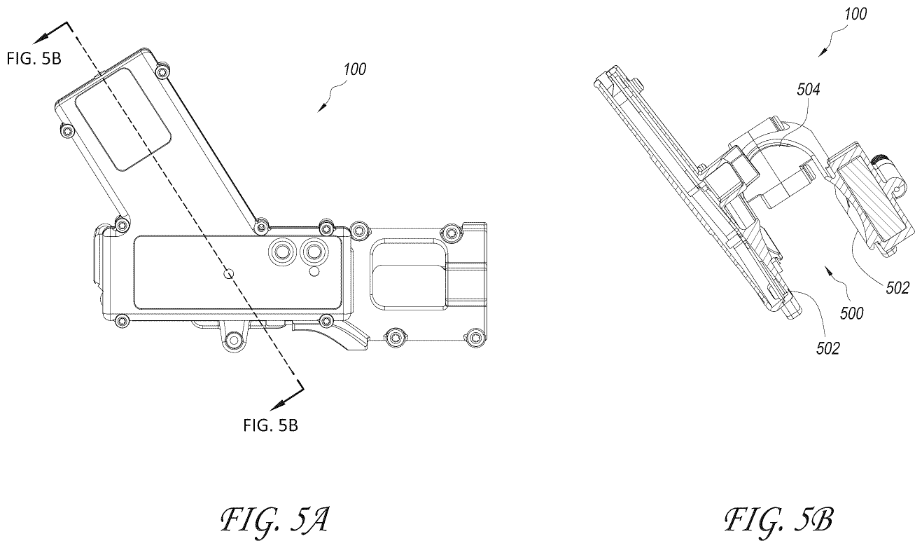

The securement portion 210 may serve to removably attach the removable treatment management apparatus 100 to the treatment injection device 110. FIG. 5B illustrates a cross section of the securement portion 210, as indicated in FIG. 5A. The securement portion 210 may be shaped so as to attach to the treatment injection device 110 in a region and in a manner that permits a user to access all or virtually all user interface elements of the treatment injection device 110 without obstruction or without appreciable obstruction. In addition, the securement portion 210 may generally be configured to be removably attachable to a treatment injection device 110 such that various components of the removable treatment management apparatus 100, such as the actuation monitor 230, can be positioned to interact with an actuator of the treatment injection device 110.

The securement portion 210 may in some embodiments include a channel 500 with a plurality of sidewalls 502. The width of the channel 500 may correspond to the distance between the respective sidewalls 502. The width of the channel 500 can be configured to be generally complimentary to the thickness of a portion of the treatment injection device(s) 110 on which the securement portion 210 is configured to attach. In some embodiments, the natural width of the channel 500 is approximately the same size or slightly smaller than the thickness of a region, such as central region, of the outside housing of a treatment injection device 110.

All or part of the securement portion 210, such as one or more of the plurality of sidewalls 502, can be made of a material (e.g., a polymer or a silicone) that is sufficiently flexible to permit deformation (e.g., compression, bending, or stretching) of one or more sidewalls 502, so that the channel 500 can be temporarily widened while sliding the removable treatment management apparatus 100 onto a treatment injection device 110, but at least a portion of one or more of the sidewalls 502 can be sufficiently stiff, rigid, or resilient to urge one or more of the plurality of sidewalls 502 to return to its original position and thereby exert a gripping force against a portion of the treatment injection device 110. The material of the securement portion 210, such as the region on the inner side of the sidewalls 502 within the channel 500, can in some embodiments be somewhat slide resistant to enhance the gripping of the securement portion 210 to the communication device.

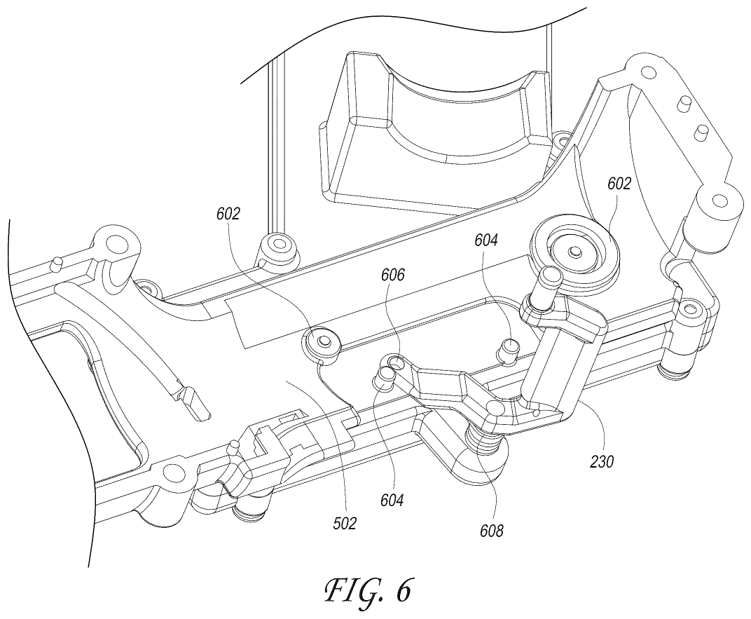

As shown in FIG. 6, the inner surface of one or more sidewalls 502 may include one or more securement structures 602 that are sized, shaped, and positioned to interact with corresponding registration structures on the treatment injection device 110. In this manner, the one or more securement structures 602 inhibit or prevent slippage or inadvertent removal of the removable treatment management apparatus 100 from the treatment injection device 110. In some embodiments, a registration structure of the treatment injection device 110 may be a surface of a fastener, such as a head of a bolt or screw. A securement structure 602 may be positioned on an inner surface of a sidewall 502 so as to interact with the registration structure. For example, the securement structure 602 may include an outer ridge configured to grip, register against, or otherwise interact with the outer rim of the fastener head of the registration structure. As another example, the securement structure 602 may include a substantially centrally-located protrusion configured to be inserted into, register against, or otherwise interact with a central recess of the fastener head of the registration structure (e.g., the hex hole of a bolt). A removable treatment management apparatus 100 or a single sidewall 502 thereof may include any number of securement structures 602.

In some embodiments, the gripping force and/or slippage resistance can reduce or eliminate the need to attach a permanent or temporary mounting component to the communication device. For example, the removable treatment management apparatus 100 can be repeatedly attached to and removed from a treatment injection device 110 without requiring (though not prohibiting) installation on the treatment injection device 110 of a separate permanently or semi-permanently attached mounting component such as a bracket, magnet, adhesive, or other fastening component, thereby providing ease of installation while preserving the original overall shape, appearance, functionality, compatibility, and/or feel of the treatment injection device 110 when the removable treatment management apparatus 100 is removed.

The securement portion 210 can in some embodiments include an upper wall 504 that can be shaped to generally correspond to the shape of an upper outer surface of a treatment injection device 110. The upper wall, 504 can provide one or more additional securement structures 602 and/or may be generally complementary in shape to the upper surface of the treatment injection device 110. As illustrated, the upper wall 504 may have a generally concave curvilinear shape for use with a treatment injection device 110 having a complementary convex curvilinear shape. The securement portion 210 can also include one or more internal surface contours configured to generally avoid interference with one or more external features on a treatment injection device 110.

The animal detector 220 can be configured to interact with a body surface of an animal 130 at an injection spot, and generate a signal indicating the removable treatment management apparatus 100 has been placed against the body surface. In some embodiments, the removable treatment management apparatus 100 may include one or more animal contact sensors 224, and the animal detector 220 may include one or more trigger objects 222 that interact with or otherwise trigger the one or more animal contact sensors 224 to generate an animal detection signal indicating placement of the removable treatment management apparatus 100 against the body surface. For example, an animal contact sensor 224 may be a Hall effect sensor, and the trigger object 222 may be a magnet. When the animal detector 220 is pressed against an injection site, the animal detector 220 may be retracted toward the body of the removable treatment management apparatus 100, and the trigger object 222 may contact or come in sufficiently close proximity to an animal contact sensor 224 that the animal contact sensor 224 detects the trigger object 222. The animal contact sensor 224 may then generate a signal indicating detection of the animal detector 220 in contact with the body surface of the animal 130.

In some embodiments, the animal detector 220 may include multiple trigger objects 222 positioned to increase the likelihood that at least one trigger object 222 will come in contact with or be placed in proximity with an animal contact sensor 224 when the animal detector 220 is pressed against an animal 130. For example, two trigger objects 222 may be positioned on opposite sides of a rotatable animal detector 220. In some embodiments, the removable treatment management apparatus 100 may include multiple animal contact sensors 224 positioned to increase the likelihood that a trigger object 222 will come in contact with or be placed in proximity with at least one animal contact sensor 224 when the animal detector 220 is pressed against an animal 130. For example, two animal contact sensors 224 may be positioned on opposite sides of a substantially circular opening through which a portion of the animal detector 220 is retracted when pressed against an animal 130.

The actuation monitor 230 can be configured to interact with an actuator of a treatment injection device 110 when a user administers a treatment using the treatment injection device 110 with the removable treatment management apparatus 100 removably attached. The actuation monitor 230 can generate a signal indicating the actuator (e.g., a trigger) of the treatment injection device 110 has been actuated (e.g., pulled). In some embodiments, one or more sidewalls 502 of the removable treatment management apparatus 100 may include one or more actuation sensors 604, and the actuation monitor 230 may include one or more trigger objects 606 that interact with or otherwise trigger the one or more actuation sensors 604 to generate an actuation signal. For example, an actuation sensor 604 may be a Hall effect sensor, and the trigger object 606 may be a magnet. When the actuator of the treatment injection device 110 is actuated (e.g., when the trigger is pulled), actuation of the actuator may case the actuation monitor 230 to pivot about a pivot point 608. This motion may cause the trigger object 606 to contact or come in sufficiently close proximity to an actuation sensor 604 that the actuation sensor 604 detects the trigger object 606. The actuation sensor 604 may then generate a signal indicating detection of the actuation event. In some embodiments, when the actuator of the treatment injection device 110 is actuated (e.g., when the trigger is pulled), the motion of the actuation monitor 230 may cause the trigger object 606 to lose contact or move a sufficiently far distance from the actuation sensor 604 that the actuation sensor 604, which had detected the trigger object 606 before actuation, now fails to detect the trigger object 606. The actuation sensor 604 may then generate a signal indicating detection of the actuation event based on this movement of the trigger object 606 away from the actuation sensor 604.

When there are multiple actuation sensors 604, the combination of actuation sensors 604 may be used to detect and distinguish between full actuation events and partial actuation events of the treatment injection device 110. In some embodiments, a first actuation sensor 604 can detect the trigger object 606 when the actuator of the treatment injection device 110 is in an unactuated position or state (e.g., the trigger is at rest, not currently depressed), and a second actuator sensor 604 can detect the trigger object 606 when the actuator of the treatment injection device 110 is in an actuated state (e.g., the trigger has been fully depressed). The first actuation sensor 604 can generate event data when the trigger object 606 is detected, event data when the trigger object 606 is no longer detected, and event data when the trigger object 606 is detected again. In a complementary manner, the second actuation sensor 604 can generate event data when the trigger object 606 is not detected, event data when the trigger object 606 is detected, and event data when the trigger object 606 is no longer detected again. When first actuation sensor 604 generates event data indicating the trigger object 606 is no longer detected and subsequent event data indicating the trigger object 606 is detected again, a determination of whether the actuation was a full actuation event or a partial actuation event can depend upon event data generated by the second actuation sensor 604. If the second actuation sensor 604 does not generate any event data representing detection of the trigger object 606 at a point in time between when the first actuation sensor 604 no longer detected the trigger object 606 and then subsequently detected the trigger object 606 again, then the actuation may be determined to be a partial actuation event. In contrast, if the second actuation sensor 604 does generate event data representing detection of the trigger object 606 at a point in time between when the first actuation sensor 604 no longer detected the trigger object 606 and subsequently detected the trigger object 606 again, then the actuation may be determined to be a full actuation event.

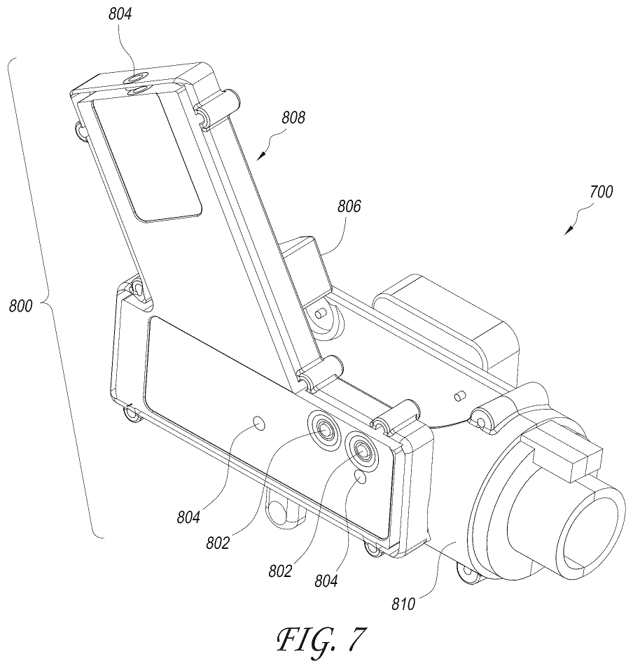

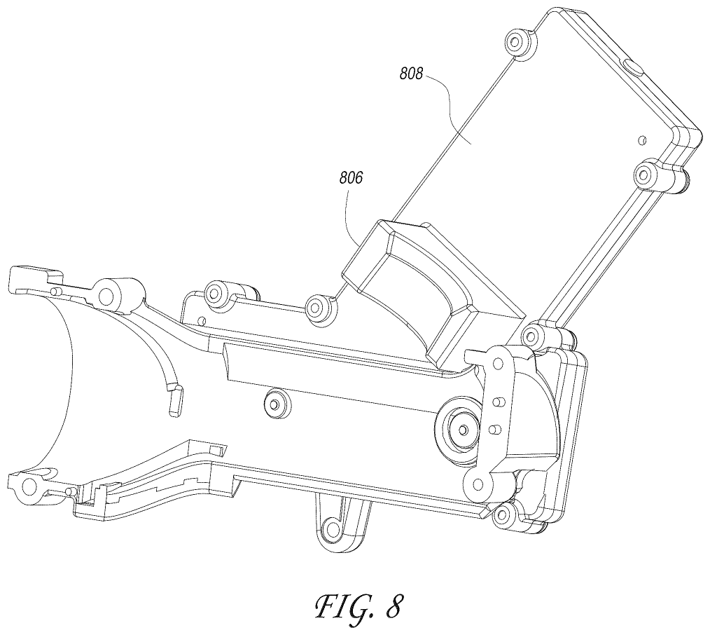

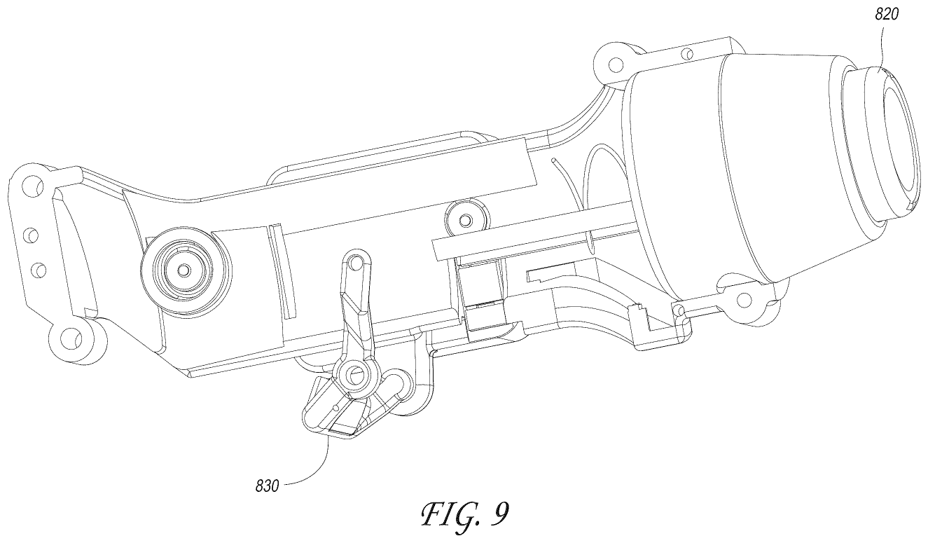

FIGS. 7, 8, 9, 10A, and 10B are various views of another example of a removable treatment management apparatus 700 and various components thereof according to some embodiments. As shown, the removable treatment management apparatus 700 includes an electronic portion 800, a securement portion 810, an animal detector 820, and an actuation monitor 830.

The electronic portion 800 may include any or all of the components and may provide any or all of the features of the electronic portion 800. For example, the electronic portion 800 may include one or more actuator components 802, indicator components 804, other components, or any combination thereof.

A protrusion of the electronic portion 800 may house a vial detector 806 positioned to be in contact with or close proximity to a vial 112 when the vial 112 is properly installed in the treatment injection device 110. In some embodiments, the vial detector 806 may be or include an inductive sensor, and vials 112 may have metal bands or caps. When a vial 112 is seated properly in the treatment injection device 110, the inductive sensor may sense the metal band or cap of the vial 112 and generate a signal regarding the vial detection event. In response to the vial detection event, one or more operations may be initiated. For example, and indicator component 804 may be activated to inform a user that the vial has been inserted correctly. As another example, a treatment tag detector 808 may be activated to detect and read a treatment tag affixed to or integrated within the vial 112. By waiting until a vial 112 has been inserted property before activation of the treatment tag detector 808, battery power may be preserved in comparison with continuously or intermittently activating the treatment tag detector 808 in the absence of detection of a vial 112.

The removable treatment management apparatus 700 may include an animal detector 820 that operates differently in some respects than the animal detector 220. As shown, the contact surface of the animal detector 820 may be significantly smaller than the contact surface of the animal detector 220 (e.g., less than about half or less than about a quarter of the contact surface of animal detector 220). Alternatively, or in addition, the distance that the exposed portion or the animal detector 820 travels before it is fully depressed and triggers an animal detection event may be significantly smaller than the distance that the exposed portion of the animal detector 220 travels (e.g., than about half or less than about a quarter of distance traveled by the animal detector 220). These differences, individual and/or in combination, may advantageously reduce or avoid the degree of lateral or counter-axis movement of the animal detector 820—in comparison with the animal detector 220—when the animal detector 820 is in contact with and pressed against the skin of the animal 130, particularly if the animal 130 is large and/or moving. Reduction in the degree of lateral movement can help to avoid bending and/or breakage of a needle 1000 of the treatment injection device 110.

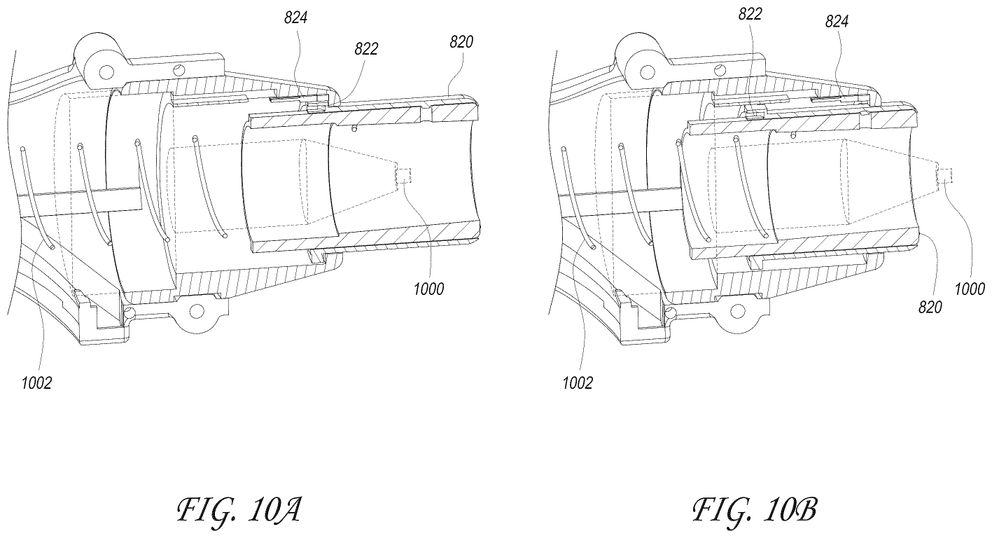

FIG. 10A illustrates the animal detector 820 in a default non-depressed position. A resilient component, such as a spring 1002, may urge the animal detector 820 away from the body of the removable treatment management apparatus 700. In this position, the needle 1000 of the treatment injection device 110 is within the nose of the removable treatment management apparatus 700 and not exposed.

FIG. 10B illustrates the animal detector 820 in a depressed position in which the animal detector 820 has been retracted at least partially within the nose of the 700, thereby exposing the needle 1000 (or, in needless administration devices, exposing the administration component to the animal).

In some embodiments, the removable treatment management apparatus 700 may include one or more animal contact sensors 824, and the animal detector 820 may include one or more trigger objects 822 that interact with or otherwise trigger the one or more animal contact sensors 824 to generate an animal detection signal. For example, an animal contact sensor 824 may be a Hall effect sensor, and the trigger object 822 may be a magnet. In the default non-depressed position shown in FIG. 10A, a trigger object 822 may be in contact with or in close enough proximity to an animal contact sensor 824 such that the animal contact sensor 824 detects the trigger object 822. This detection can be confirmation that the animal detector 820 is in the default non-depressed position. When the animal detector 820 is pressed against an injection site, the animal detector 820 may be retracted toward the body of the removable treatment management apparatus 700, and the trigger object 822 may lose contact or otherwise move sufficiently far from the animal contact sensor 824 that the animal contact sensor 824 no longer detects the trigger object 822. The animal contact sensor 824 may then generate a signal indicating detection of the animal detector 820 in contact with the body surface of the animal 130.

### Example Treatment Management System Processing

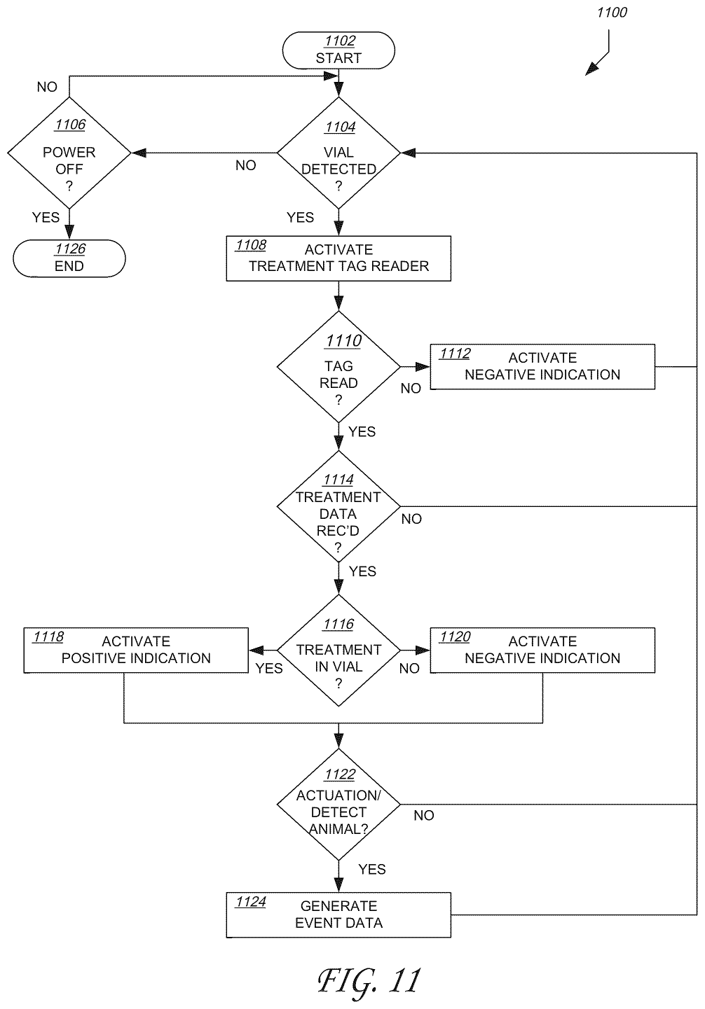

FIG. 11 is a flow diagram of an illustrative routine 1100 that may be executed by a removable treatment management apparatus to manage and track administration of treatment from a treatment injection device 110. The routine 1100 begins at block 1102. The routine 1100 may begin in response to an event, such as when the removable treatment management apparatus is powered on or removably attached to a treatment injection device 110. When the routine 1100 is initiated, a set of executable program instructions stored on one or more non-transitory computer-readable media (e.g., hard drive, flash memory, removable media, etc.) may be loaded into memory (e.g., random access memory or RAM) of the removable treatment management apparatus, such as a processor and memory on a PCB within an electronic portion of the removable treatment management apparatus. In some embodiments, the routine 1100 or portions thereof may be implemented on multiple processors, serially or in parallel.

At decision block 1104, the removable treatment management apparatus may determine whether a vial 112 has been inserted into the treatment injection device 110. The determination may be based on data generated by the vial detector 806 as described in greater detail above. If a vial 112 has been inserted, the routine 1100 may proceed to block 1108. Otherwise, if a vial 112 has not been inserted, the routine 1100 may proceed to decision block 1106.

At decision block 1106, if a vial 112 has not been detected and the removable treatment management apparatus has been powered off, the routine 1100 may terminate at block 1126. Otherwise, the routine 1100 may return to decision block 1104 to monitor for insertion of a vial 112 into the treatment injection device 110.

At block 1108, if insertion of a vial 112 has been detected, the removable treatment management apparatus may activate the treatment tag detector 808 in response to proper insertion of a vial 112 into the treatment injection device 110. Waiting until proper insertion of a vial 112 to activate the treatment tag detector 808 can help to preserve battery life for the removable treatment management apparatus 100, as the treatment tag detector 808 may in some cases be one of the larger draws on batter power on a per-operation or per-time-unit basis.

At decision block 1110, the removable treatment management apparatus may determine whether the treatment tag detector 808 successfully read a treatment tag 302 on the vial 112. If so, the routine 1100 may proceed to decision block 1114. Otherwise, if a treatment tag 302 has not been read successfully, the routine 1100 may proceed to block 1112.

At block 1112, if a treatment tag 302 has not been read successfully, the removable treatment management apparatus may activate an indicator component (e.g., indicator component 204 or indicator component 804) to indicate, to a user, that the vial 112 may need to be rotated so that the treatment tag 302 may be placed adjacent to or sufficiently close to the treatment tag detector 808. The routine 1100 may then return to decision block 1104 or otherwise wait for a repositioning of the vial 112 or detection of a treatment tag 302.

At decision block 1114, the removable treatment management apparatus may determine whether treatment data has been received from a user system 102. In some embodiments, treatment data may consist of or include an animal identifier so that any events of the treatment injection device 110 can be associated with a particular animal. In some embodiments, the treatment data may also or alternatively consist of or include a treatment identifier so that the removable treatment management apparatus can determine whether the proper treatment is currently installed in the treatment injection device 110. If treatment data is received, the routine 1100 may proceed to decision block 1116. Otherwise, if treatment data has not been received, the removable treatment management apparatus may continue to monitor for receipt of treatment data and/or the routine 1100 may return to some prior block such as decision block 1104.

In some embodiments, event data may be generated at block 1124 (described below) even if at decision block 1114 no treatment data is received. For example, a user can still administer treatment from a treatment injection device, and the removable treatment management apparatus can still use the animal detector to determine whether an animal is detected, the actuation monitor to detect a partial or full trigger pull, etc. In this instance, the removable treatment management apparatus can still send event data to the user system. The event data may not include an animal ID (see Table 1), but the event data may still be used to record administration of a dose of treatment to an unknown animal.

At decision block 1116, the removable treatment management apparatus may determine whether the treatment in vial 112 currently inserted into the treatment injection device 110 is the same as the treatment identified in the treatment data. In some embodiments, the determination may be based on a comparison of data obtained from the treatment tag 302 (e.g., a treatment identifier of the treatment in the vial 112) and the treatment identifier included in treatment data. If the treatment identifiers match, then the removable treatment management apparatus can determine that the treatment in the vial 112 is the same as the treatment identified in the treatment data. In this case, the routine 1100 may proceed to block 1118. Otherwise, if the identifiers do not match or the removable treatment management apparatus otherwise determines that the treatment in the vial 112 is different than the treatment identified in the treatment data, the routine 1100 may proceed to block 1120. In some embodiments, if the identifiers do not match or the removable treatment management apparatus otherwise determines that the treatment in the vial 112 is different than the treatment identified in the treatment data, no indication may be given to the user and the routine 1100 may proceed to decision block 1122.

At block 1118, if the indicated treatment is in the currently-installed vial, the removable treatment management apparatus may cause an indicator component 204 to indicate that the removable treatment management apparatus is to be used or may be used to administer the indicated treatment to the current animal. For example, an indicator component 204 may be illuminated a particular color (e.g., green) and/or may be illuminated in a particular pattern (e.g., solid).

At block 1120, if the indicated treatment is not in the currently-installed vial, the removable treatment management apparatus may cause an indicator component 204 to indicate that the removable treatment management apparatus is not to be used or otherwise is not configured to administer the indicated treatment to the current animal. For example, an indicator component 204 may be illuminated a particular color (e.g., red) and/or may be illuminated in a particular pattern (e.g., blinking). In some embodiments, this may not prevent use of the treatment injection device 110 to nevertheless administer treatment.

At decision block 1122, the removable treatment management apparatus may determine whether one or more animal detector events and/or actuation monitor events have occurred. If so, the routine 1100 may proceed to block 1124. Otherwise, the routine 1100 may return to a prior block, such as decision block 1104.

At block 1124, the removable treatment management apparatus may generate data regarding the event(s) detected. In some embodiments, event data may be generated at block 1124 even if at decision block 1114 no treatment data is received, and/or even if at decision block 1116 a determination is made that the treatment in the vial currently inserted into the treatment injection device 110 is not the same as the treatment identified in the treatment data.

Examples of various events and corresponding event data transmissions-both to and from the removable treatment management apparatus—are provided in Table 1 below. The example events and event data described in Table 1 and elsewhere herein are provided for illustrative purposes only, and are not intended to be limiting, required, or exhaustive.

Examples of various events and corresponding event data transmissions-both to and from the removable treatment management apparatus—are provided in Table 1 below. Example bit locations and values for signaling the status of various sensors in a 1-byte “Sensor Status” data item are provided in Table 2. The example events and event data described in Table 1, Table 2, and elsewhere herein are provided for illustrative purposes only, and are not intended to be limiting, required, or exhaustive

TABLE 1
Example Event Data Transmissions

1. Vial Inserted
a. Notification sent to user system.
b. 1-byte; See “Sensor Status”
characteristic for values.
2. Vial ID scanned
a. Initiated by detection of new vial.
b. Notification sent to user system.
c. 12-byte array.
3. Set error state written by user system
a. 1-byte
b. 0x00: Status OK
c. 0x01: Error
4. Animal ID written by user system
a. Sent as new animal is identified.
b. 32-bit unsigned integer.
5. Injection needed written by user system
a. Sent if animal needs injection from this
treatment injection device.
b. 1-byte; 0x01 if injection is needed.
c. Turns LED solid green.
d. Will be reset after injection or timeout
(TBD seconds).
6. Animal detector depressed
a. Notification sent to user system when animal
detector fully depressed.
b. 1-byte; See “Sensor Status”
characteristic for values.
7. Trigger pulled
a. Notification sent to user system when
trigger fully pulled.
b. 1-byte; See “Sensor Status”
characteristic for values.
8. Trigger Released
a. Notification sent to user system when
trigger fully released.
b. 1-byte; See “Sensor Status”
characteristic for values.
9. Animal detector released
a. Notification sent to user system when
guard is no longer fully depressed.
b. 1-byte; See “Sensor Status”
characteristic for values.
10. Injection record saved to memory.
a. Bytes 0-3: Animal ID
b. Bytes 4-15: Vial ID
c. Bytes 16-19: Epoch Timestamp (seconds)
d. Byte 20: Full trigger pulls
e. Byte 21: Partial trigger pulls
f. Notification sent out to user system.
g. Length: 22 bytes
h. Updated to next injection record when
record processed is written to.
11. Record Processed written by user system
a. Signals to firmware that above injection
record has been handled by the user
system and is safe to delete.
b. Length: 22 bytes; value must match
Injection record above.
12. Current time synchronization
a. Read current time by user system.
b. The value is updated at 5 second intervals
by firmware.
c. If written to by user system, time in
treatment injection device is updated to same
value.

TABLE 2
Example Sensor Status

| Bit | 7 | 6 | 5 | 4 | 3 | 2 | 1 | 0 |
|---|---|---|---|---|---|---|---|---|
| Trigger release sensor off | X | X | X | X | X | X | X | 0 |
| Trigger release sensor on | X | X | X | X | X | X | X | 1 |
| Trigger pull sensor off | X | X | X | X | X | X | 0 | X |
| Trigger pull sensor on | X | X | X | X | X | X | 1 | X |
| Needle guard depressed sensor off | X | X | X | X | X | 0 | X | X |
| Needle guard depressed sensor on | X | X | X | X | X | 1 | X | X |
| Vial presence sensor off | X | X | X | X | 0 | X | X | X |
| Vial presence sensor on | X | X | X | X | 1 | X | X | X |

### Terminology

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A treatment management apparatus for managing treatment administered to livestock, the treatment management apparatus comprising:
a wireless communication interface configured to receive, from a user system, treatment data representing an animal to be treated and a treatment to be administered;
an animal detector configured to detect placement of the treatment management apparatus against a surface of the animal; and
an actuation monitor configured to detect actuation of a treatment initiation component to initiate administration of the treatment of the animal;
wherein the wireless communication interface is further configured to send, to the user system, administration data regarding the administration of the treatment, wherein the administration data represents one or more events associated with the animal detector and the actuation monitor.

2. The treatment management apparatus of claim 1, further comprising a treatment tag detector configured to read a treatment tag affixed to a vial.

3. The treatment management apparatus of claim 2, further comprising a vial detector configured to detect installation of the vial, wherein the treatment tag detector is configured to activate the treatment tag detector in response to detecting installation of the vial.

4. The treatment management apparatus of claim 3, further comprising an indicator component configured to indicate whether the vial includes the treatment represented by the treatment data received from the user system.

5. The treatment management apparatus of claim 1, further comprising a securement portion configured to removably attach to a treatment injection device, wherein the actuation monitor is configured to detect movement of an actuator of the treatment injection device.

6. The treatment management apparatus of claim 5, wherein the securement portion comprises a first sidewall and a second sidewall forming a channel, wherein to removably attach the treatment management apparatus to the treatment injection device, the treatment injection device is inserted into the channel.

7. The treatment management apparatus of claim 6, wherein an interior surface of the first sidewall comprises a securement structure configured to interact with a registration structure of the treatment injection device, wherein interaction of the securement structure with the registration structure reduces slippage of the treatment management apparatus.

8. The treatment management apparatus of claim 5, wherein the actuation monitor pivots at a pivot point when the actuator of the treatment injection device is moved.

9. The treatment management apparatus of claim 8, wherein the actuation monitor comprises a trigger object, wherein an inner surface of the securement portion comprises an actuation sensor configured to detect the trigger object.

10. The treatment management apparatus of claim 9, wherein the actuation sensor comprises a Hall effect sensor.

11. The treatment management apparatus of claim 9, wherein the inner surface of the securement portion comprises a second actuation sensor configured to detect the trigger object, wherein detection of the trigger object by the actuation sensor corresponds to actuation of the actuator of the treatment injection device, and wherein detection of the trigger object by the second actuation sensor corresponds to positioning of the actuator of the treatment injection device in an unactuated position.

12. The treatment management apparatus of claim 5, wherein the wireless communication interface is configured to generate the treatment data in response to detecting, using the actuation monitor, movement of the actuator of the treatment injection device.

13. The treatment management apparatus of claim 5, wherein the animal detector comprises a trigger object, wherein an inner surface of the securement portion comprises an animal contact sensor configured to detect the trigger object.

14. The treatment management apparatus of claim 5, wherein the wireless communication interface is configured to generate the treatment data in response to detecting, using the actuation monitor, the movement of the actuator of the treatment injection device and detecting, using the animal detector, the treatment management apparatus against a surface of the animal.

15. The treatment management apparatus of claim 5, wherein the wireless communication interface is configured to generate second treatment data in response to detecting, using the actuation monitor, the movement of the actuator of the treatment injection device and failing to detect, using the animal detector, the treatment management apparatus against a surface of the animal, wherein the second treatment data represents one of a priming operation or a wasted treatment administration operation.

16. The treatment management apparatus of claim 5, wherein the treatment management apparatus is fixable, removeable, and reusable.

17. A system comprising:
a treatment management apparatus for use with a treatment injection device for managing treatment administered to livestock using the treatment injection device, the treatment management apparatus comprising:
an actuation monitor configured to detect movement of an actuator of a treatment injection device;
a treatment tag detector configured to read a treatment tag affixed to a vial installed in the treatment injection device; and
a vial detector configured to detect installation of the vial into the treatment injection device; and
an executable treatment administration manager configured to cause a user system to communicate with the treatment management apparatus.

18. The system of claim 17, wherein the executable treatment administration manager is further configured to cause the user system to communicate with an application server regarding treatment regimens to be implemented using the treatment management apparatus.

19. The system of claim 17, further comprising an animal identification device configured to read a tag affixed to an animal and transmit, to the user system, animal identification data read from the tag.

20. The system of claim 17, wherein the treatment management apparatus further comprises a securement portion configured to removably attach to the treatment injection device.

21. The system of claim 20, wherein the treatment management apparatus is fixable, removeable, and reusable.

22. A treatment management apparatus for use with a treatment injection device for managing treatment administered to livestock using the treatment injection device, the treatment management apparatus comprising:
a securement portion configured to be fixed to a treatment injection device;
an actuation monitor configured to detect movement of an actuator of the treatment injection device; and
an electronic portion comprising:
a treatment tag detector configured to read a treatment tag affixed to a vial installed in the treatment injection device;
a vial detector configured to detect installation of the vial into the treatment injection device, wherein the treatment tag detector is configured to activate the treatment tag detector in response to detecting installation of the vial; and
a wireless communication interface configured to:
receive, from a user system, treatment data representing an animal to be treated and a treatment to be administered; and
send, to the user system, administration event data representing one or more events associated with the animal and the treatment.
